(19) United States
(12) Patent Application Publication
(10) Pub. No.: US 2015/0182634 A1
(43) Pub. Date: Jul. 2, 2015

(54) Molecular Design and Chemical Synthesis of Pharmaceutical-Ligands and Pharmaceutical-Pharmaceutical Analogs with Multiple Mechanisms of Action

(71) Applicants: Cody P. Coyne, Starkville, MS (US); Ryan Bear, Starkville, MS (US); Toni Jones, Mathiston, MS (US)

(72) Inventors: Cody P. Coyne, Starkville, MS (US); Ryan Bear, Starkville, MS (US); Toni Jones, Mathiston, MS (US)

(21) Appl. No.: 13/998,960

(22) Filed: Dec. 27, 2013

(51) Int. Cl. A61K 47/48; A61K 31/706; C07D 471/04; C07H 19/16; C07H 19/06; C07H 19/10; A61K 31/704; A61K 31/7076

(52) U.S. Cl. CPC A61K 47/48561; A61K 31/704; A61K 31/706; A61K 47/484; A61K 31/7076; A61K 47/48276; C07H 19/16; C07H 19/06; C07H 19/10; C07D 471/04

(57) ABSTRACT

Multi-phase and single-phase chemical reaction schemes have been developed for the synthesis of pharmaceutical-ligand analogs, pharmaceutical-pharmaceutical analogs, and similar molecular-molecular analogs that possess multiple mechanisms of action. The multi-phase organic chemical reaction schemes include relatively mild reaction conditions, high end product yields, and comparatively rapid completion of chemical reactions, which are all of particular utility for the synthesis of preparations including covalent pharmaceutical-receptor ligand or pharmaceutical-immunoglobulin analogs. Examples of pharmaceutical-ligand preparations that can be synthesized utilizing the multi-step chemical reaction schemes include covalent chemotherapeutic-ligand agents that possess selective targeted delivery properties and a capacity to exert additive and synergistic levels of cytotoxic anti-neoplastic potency. Pharmaceutical-pharmaceutical analogs, including chemotherapeutic-chemotherapeutic analogs that are capable of exerting multiple mechanisms of action, can be synthesized using either of the described multi-phase or single-phase organic chemistry reaction schemes. Each of these representative examples has utility against a spectrum of disease states including, for example, neoplastic conditions such as mammary adenocarcinoma/carcinoma, ovarian carcinoma, prostatic carcinoma, intestinal carcinoma, melanoma, leukemia, myeloma, and lymphoma.

### CROSS-REFERENCE TO RELATED APPLICATION

This application claims priority from U.S. Provisional Patent Application Ser. No. 61/848,239 filed Dec. 28, 2012. The entirety of that provisional application is incorporated herein by reference.

### FIELD OF THE INVENTION

The present invention relates to the molecular design and chemical synthesis of pharmaceutical-ligand analogs, pharmaceutical-pharmaceutical analogs, and similar molecular-molecular analogs with multiple mechanisms of action. More specifically, the invention provides novel methodologies for single-phase and multi-phase syntheses for the production of such analogs.

### BACKGROUND OF THE INVENTION

### Description of Prior Art: Chemistry

Despite early diagnostic surveillance, improvements in imaging instrumentation, advances in image processing, and better understanding of breast cancer cell biology, about 30% of patients with early-stage breast cancer have recurrent disease. In general, systemic agents are active at the beginning of therapy for 90% of primary breast cancer lesions while 50% of metastases are resistant to therapy, which is an occurance that is not only common but anticipated. Resistance to conventional chemotherapy is frequently associated with the relative over-expression of P-glycoprotein (MDR-1) and multi-drug resistance protein-1 (MRP-1). Often simultaneous with chemotherapeutic-resistance and P-glycoprotein expression, 25-30% of primary breast carcinomas also demonstrate HER2/neu gene amplification and HER2/neu over-expression where HER2/neu membrane densities can approach 1×10⁶ or more per cell. In addition to primary breast carcinoma, HER2/neu over-expression occurs in 15-30% of ovarian carcinomas and in conditions of gastric carcinoma.

The human epidermal growth factor receptor 2 complex (HER2/neu, ErbB2, CD340) is a 185-kDa trans-membrane glycoprotein that is a product of the c-erbB-2 (HER2/neu) pro-oncogene located on the 17q21 chromosome. By classification, HER2/neu is a member of the epidermal growth factor (ErbB) receptor family that includes EGFR, HER2, HER3 and HER4 which function as surface membrane-associated tyrosine kinases involved in signal transduction where HER2/neu is an essential mediator of cellular proliferation, differentiation, and survival. Natural binding ligands for HER2/neu to date have not been identified, but the tyrosine kinase activity of HER2/neu is stimulated by HER2/neu homodimerization (enhanced by HER2/neu over-expression), or heterdimerization with other members of the EGFR receptor family. The biological impact of HER2/neu over-expression is an elevated sensitivity to growth factor stimulation and suppression of negative regulatory mechanisms involved in signal attenuation. Such responses directly correlate with HER2/neu over-expression profiles known in clinical oncology to be closely associated with aggressive growth behavior, disease reoccurance, poor long-term prognosis, and chemotherapeutic-resistance.

Similar to HER2/neu, over-expression of EGFR is recognized in approximately 25-60% of mammary carcinomas where it can reach expression densities of approximately 2.2×105 per cell. Both increased copy number and over-expression of EGFR are associated with high tumor grade, greater patient age, large residual tumor size, high proliferation index, aberrant p53, poor patient outcome, and less than optimal response to therapy. Epidermal growth factor receptor (EGFR, ErbB-1, HER1) is a member of the ErbB epidermal growth factor family of receptors. Located on the external surface membrane, EGFR1 is expressed as a 170-kDa glycoprotein with an N-linked glycan and GlcNAc terminus. The ligands, epidermal growth factor and transforming growth factor (TGFα) both activate EGFR1 that in turn results in EGFR1 monomer being converted to an activated homodimer complex. The transformation results in marked increases in intrinsic intracellular protein tyrosine kinase activity and auto-phosphorylation of tyrosine residues. Such changes initiate down-stream activation and signaling of several proteins that in turn promote induction of MAPK, Akt, and JNK signal transduction cascades ultimately leading to DNA synthesis and increased cellular proliferation. Mutations of EGFR1 over-expression foster continual stimulation and patterns of uncontrolled cellular division.

In neoplastic conditions that uniquely or over-express HER2/neu or EGFR the administration of anti-HER2 and anti-EGFR monoclonal immunoglobulin effectively slows neoplastic cell proliferation rates. Anti-EGFR blocks continued ligand-mediated EGFR stimulation while both anti-HER2/neu and anti-EGFR both promote receptor down-regulation. One biological effect anti-HER2/neu (tratuzumab) is a significant suppression of neoplastic cell proliferation in part through inhibition of p27-regulated proliferation. Related monoclonal immunoglobulin preparations (e.g. pertuzumab) bind to a different HER2/neu epitope and inhibit HER2-HER3 receptor heterodimerization.

Pharmacology and Organic Chemistry Reactions: Due in large part to their chemical composition, molecular configuration and wide spectrum of anti-neoplastic potency, the anthracyclines have traditionally been the chemotherapeutic class most commonly bonded covalently to molecular platforms can facilitate “selective” targeted delivery. The spectrum of anthracylines utilized to synthesize covalent anthracycline-immunochemotherapeutics to date has largely included doxorubicin and to a lesser extent daunorubicin and epirubicin. A relatively small collection of semi-synthetic heterobifunctional organic chemistry reactions have previously been developed for covalently bonding anthracycline-class chemotherapeutics to biologically active protein fractions including monoclonal immunoglobulin.

One common methodology for the semi-synthesis of anthracycline conjugates involves the creation of a covalent bond at the C₃ α-monoamine group of the anthracycline carbohydrate moiety. Methodologies of this type include those that utilize oxidized dextran as a molecular bridge where their aldehyde groups are reacted with both the C₃ α-monoamine group of the anthracycline carbohydrate moiety, and the amine group of immunoglobulin ε-lysine amino acid residues. In an analogous semi-synthesis method, the anthracycline C₃ α-monoamine group is enzymatically conjugated to oxidized aldehydes of immunoglobulin galactose moiety yielding a Schiff base. Glutaraldehyde can similarly be used as another type of molecular “bridge” where it forms covalent bonds at the C₃ α-monoamine of the anthracycline carbohydrate moiety. In the use of either dextran or glutaraldehyde, it is critical that reagent concentrations and reactant molar ratios be optimized and reaction times are carefully monitored to avoid formation of aberrant lower-potency side products. In this context, a disadvantage associated with each of these two methods it that the chemical reactions lack significant selectivity and they can be difficult to control leading to a number of side reactions that ultimately requires the removal of undesirable side products and relatively low end-product yield. Analogs of cis-aconitic anhydride can also be used to semi-synthesize acid-labile anthracycline-immunoconjugates resulting in the formation of a pH-sensitive covalent bond at the C₃ α-monoamine doxorubicin and daunorubicin accompanied by the production of covalent bonds at immunoglobulin ε-lysine amino acid residues similar to dextran and glutaraldehyde. Similarly, a versatile method for synthesizing anthracycline-immunoconjugates utilizes the organic polymer, N-(2-hydroxypropyl)-methacrylamide (HPMA) to form a covalent bond with doxorubicin through either a N-cis-aconityl reaction at the C₃ α-monamine, or by formation of a hydrazone bond at the (C₁₃-keto) position.

Succinimidyl-4-(N-maleimidomethyl)cyclohexane-1-carboxylate (SMCC) has been utilized as a heterobifunctional covalent cross-linking reagent for the synthesis of covalent anthracycline immunochemotherapeutic agents. Due to its labile nature in aqueous buffers, the N-hydroxysuccinimide (NHS) group of succinimidyl-4-(N-maleimidomethyl)cyclohexane-1-carboxylate (SMCC) ideally needs to first be reacted with the C₃ α-monoamide group of anthracyclines followed by subsequent reaction of the SMCC maleimide group with available sulfhydryls of N-succinimidyl-S-acetylthioacetate (SATA) chemically introduced into immunoglobulin fractions at ε-lysine amino acid residues. Attributes of utilizing succinimidyl-4-(N-maleimidomethyl)cyclohexane-1-carboxylate (SMCC) as a heterbifunctional covalent cross-linking reagent for the semi-synthesis of epirubicin-immunoconjugates include chemical properties that allow it to create covalent bonds in a chemically selective and controlled manner. Speculation suggests there may also be an advantage of SMCC forming a covalent bond at the C₃ α-monoamide group of the epirubicin carbohydrate moiety in contrast to the (C₁₃-keto) position. Theoretically, selective reaction of succinimidyl-4-(N-maleimidomethyl)cyclohexane-1-carboxylate (SMCC) with the α-monoamide group may reduce the influence of steric hindrance phenomenon during immunoconjugate-antigen complex formation. Such a spatial orientation could therefore result in improved physical interactions to develop between anthracycline moieties and doubled-stranded nuclear DNA or intracellular enzyme systems potentially involved in mediating the liberation of non-conjugated “free” anthracycline within the cytosol. The relatively more rapid, convenient and economical features of succinimidyl-4-(N-maleimidomethyl)cyclohexane-1-carboxylate (SMCC) are additional attributes that make it an attractive reagent for the semi-synthesis of epirubicin-immunoconjugates.

Relatively few investigations have described the synthesis of anthracycline immunochemotherapeutics through the creation of a covalent bond at the C₁₃-keto group of anthracyclines. In this regard, hydrazide/hydrazone chemical reactants represent an alternative method for the synthesis of covalent bonds between the C₁₃-keto group of anthracyclines and selective “targeted” delivery platforms including monoclonal immunoglobulin. Both doxorubicin and epirubicin have been covalent linked to molecular “targeting” platforms through the formation of a reactive hydrazide and the creation of an “acid-sensitive” or “acid-labile” hydrazone bond. The heterobifuctional reagent, SMCC-hydrazide can alternatively be applied in this context where the hydrazide group is reacted with the (C₁₃-keto) of doxorubicin similar to the hydrazide analogs 4[N-maleimidomethyl]cyclohexane-1 carboxyl-hydrazide; 6-maleimidocaproyl-hydrazide (3,3′-N[ε-maleimidocaproic acid] hydrazide), and N-(2-hydroxyprophyl)methacrylamide (HPMA) based analogs.

The anthracycline immunochemotherapeutics that are labile under acidic-pH conditions but molecularly stable in plasma are proposed to rapidly liberating their anthracycline moiety in the relatively lower cytosol pH of 7.0, and especially at the pH 5.0-5.5 environment of endosome/lysosome/phagolysosome encountered following internalization by mechanisms of receptor mediated endocytosis. Despite the seemingly obvious attributes of acid-labile anthracycline-aconityl-immunoglobulin immunoconjugates, some reagents and methodologies can yield preparations that liberate only 45% of their total chemotherapeutic content in an acid-labile manner. Related investigations revealed that in certain human neoplastic cell lines, anthracycline immunochemotherapeutics synthesized in this manner do not provided an elevated level of cytotoxic anti-neoplastic activity.

Other chemotherapeutics in addition to the anthracyclines have been covalently bonded to large molecular weight carrier molecules including but not restricted to monoclonal immunoglobulin and receptor ligands. One example includes gemcitabine which is a deoxycytidine nucleotide analog that functions as a chemotherapeutic when intracellularly it becomes triphosphoralated allowing it in turn to substitute for cytidine during DNA replication resulting in its incorporation into DNA strands and the inhibition of DNA polymerase activity. In contrast to covalent anthracycline conjugates, a very limited number of published reports have described the molecular design, synthesis and cytotoxic anti-neoplastic potency of gemcitabine covalent bound to selective “targeting” ligands while an even smaller number of reports have described the production and potency of covalent gemcitabine immunochemotherapeutics.

The creation of a synthetic covalent bond between gemcitabine and monoclonal immunoglobulin, immunoglobulin fragments (e.g. Fab′), receptor ligands or other biologically active protein fractions can be achieved utilizing a relatively small collection of organic chemistry reaction schemes. Generation of a covalent bond at the C₅-methylhydroxy group of gemcitabine represents one molecular approach to synthesizing covalent gemcitabine-immunochemotherapeutics or gemcitabine-ligand preparations. A second and more infrequently utilized molecular strategy involves the creation of a covalent bond at the cytosine-like C₄-amine group of gemcitabine either as a direct link to a “targeting” delivery platform or to alternatively create a gemcitabine reactive intermediate.

When necessary, the C₄—NH₂, C₃′—OH and C₅′—OH groups of gemcitabine can be reversibly protected utilizing di-tert-dibutyl dicarbonate. Few if any reports have described the molecular design and efficacy evaluation of covalent gemcitabine immunochemotherapeutics synthesized that entail the generation of a covalent bond between either at the C₅-methylhydroxy or cytosine-like C₄-amine groups of gemcitabine.

Similar molecular strategies have been employed for the synthesis of covalent anthracycline immunochemotherapeutics through the formation of a covalent bond at the α-monoamine (C₃-amine) group associated with the carbohydrate moiety of doxorubicin, daunorubicin, or epirubicin.

### Covalent Bonding of Reactive Chemotherapeutic Intermediate to Selective Targeting Platform

Methodologies that utilize oxidized dextran as a molecular bridge generate aldehyde groups that in turn reacted with the anthracycline carbohydrate moiety and the amine group of immunoglobulin ε-lysine amino acid residues. In an analogous semi-synthesis method the anthracycline C₃ α-monoamine group is enzymatically conjugated to aldehydes groups yielding a Schiff base following sodium periodate oxidation of the immunoglobulin galactose moiety. Glutaraldehyde can similarly be used as another type of molecular “bridge” where it forms covalent bonds at the carbohydrate moiety of monoclonoal immunoglobulin.

Alternatively, sulfhydryl-reactive maleimide anthracycline intermediates have been synthesized utilizing 4[N-maleimidomethyl]cyclohexane-1 carboxyl-hydrazide, 6-maleimidocaproyl-hydrazide (3,3′-N[E-maleimidocaproic acid] hydrazide), and other similar maleimide reactants. The sulfhydryl-reactive maleimide groups incorporated into these anthracyline intermediates then form a covalent bond with either cysteine amino acid residues or the thiolated ε-amine groups of lysine amino acid residues within biological protein fractions created through either DTT disulfide bond disruption, or introduction of sulfhydryl groups with vinylsulfone, 2-iminothiolane, or mercaptosuccinimide. The type of thiolation methodology applied is critically important because some reagents like iminothiolane under certain conditions promote protein-protein polymerization side-reactions.

### Description of Prior Art: Selective “Targeted” Chemotherapeutic Delivery Strategies

Selective “targeted” delivery of chemotherapeutics has been achieved applying non-protein ligands including folate (folate receptors), galactomannan/mannan* (α₂macroglobulin receptor), HPMA (N-(2-hydroxypropyl)-methacrylamide for integrin), hyaluronan* (CD44), dextran/PEG (polyethylene glycol), and D-α-tocopheryl polyethylene glycol 1000 succinate). Pharmaceuticals that have been applied as a molecular platform for selective “targeted” delivery include Tomoxifen (estrogen receptors: fluorescent 4-hydroxytamoxifen; 2-(1-hexyloxyethyl)-2-devinyl pyropheophorbide-α as a photodynamic agent, [¹¹¹In]-DTPA-tamoxifen for nuclear imaging). Biological ligands or ligand fragments that can be effective for selective “targeted” chemotherapeutic delivery include epithelial growth factor or EGF fragments (EGFR), transferrin (across BBB), alpha fetoprotein, leutinizing releasing hormone (AN152), somatostatin analog (AN162/AEZS-124), chorionic gonadotropin, gonadotropin releasing hormone, albumin and lactosaminated albumin (DOX-EMCH), non-immunoglobulin protein fractions including DNA aptamer, transferin, albumin/lactosaminated albumin or synthesized as a sulfhydryl-reactive maleimide analog that form covalent anthracycline-albumin complexes (e.g. DOXO-EMCH) following intravenous administration.

Monoclonal immunoglobulin in the form of IgG, Fab′ or F(ab′)₂ has been applied to facilitate selective “targeted” chemotherapeutic delivery for CD5⁺ (T-cell lymphoma), CD 19 (B-cell lymphoma), CD22 (non-Hodgkin lymphoma), CD30 (Hodgkin lymphoma), CD33 (acute myelogenous leukemia), CD44 (mammary carcinoma), CD74 (multiple myeloma, B-cell lymphoma), carcinoembryonic antigen or CEA (LoVo colon carcinoma), cervical carcinoma cell-surface antigen (cervical carcinoma), epidermal growth factor receptor (mammary carcinoma), epidermal growth factor receptor (metastatic melanoma), epidermal growth factor receptor (oral epidermoid carcinoma), CD44 (mertansine: squamous cell carcinoma), CD56 (mertansine: small-cell lung), CD56 (mertansine: lung cancer), CD56 (mertansine; ovarian cancer), mucin CanAg (mertansine: colorectal cancer), chondroitin sulfate proteoglycan (metastatic melanoma), epidermal growth factor receptor (EGFR, mammary adenocarcinoma/carcinoma, intestinal carcinoma, ovarian carcinoma, prostatic carcinoma); HER2/neu (epidermal growth factor 2: mammary adenocarcinoma/carcinoma); GPNMB (transmembrane glycoprotein NMB for melanoma and mammary carcinoma), 3H11 (gastric carcinoma), Lewis Y (Le^{y}) like antigen (lung adenocarcinoma L2987), Lewis Y (intra-cerebral small-cell lung carcinoma, colon carcinoma RCA, mammary carcinoma MCF-7), Midkine (hepatocellular carcinoma HepG2), OA3 surface antigen (ovarian carcinoma), PLC/PRF/5 (hepatocellular carcinoma) and HepG2 791T/36 (IgG₂ pre-targeting).

Chemotherapeutics that have been covalently bonding to large molecular weight molecules that possess properties that can facilitate selective “targeted” delivery include the anthracyclines (IgG and ligands), methotrexate/Pemetrexed (IgG), vinca alkaloids (modified vinorelbine: IgG), bleomycin (IgG and non-IgG), chlorambucil (non-IgG transferrin), gemcitabine (non-IgG), paclitaxel (non-IgG), calicheamicins (IgG), monomethyl auristatin E or MMAE (IgG), and maytansinoids (e.g. mertansine: IgG).

### Description of Prior Art: Selective “Targeted” Anthracyclines and Gemcitabin Delivery

Selectively “targeted” anthracyclines have demonstrated selective “targeted” cytotoxic anti-neoplastic potency against, metastatic melanoma (in-vitro and in-vivo), multiple myeloma (in-vitro and in-vivo), B-cell lymphoma (in-vitro and in-vivo), T-cell lymphoma (in-vivo), pulmonary carcinoma (in-vitro and in-vivo), lung carcinoma (small cell) (in-vivo), colon carcinoma (in-vitro), and hepatocarcinoma (in-vivo).

Most commonly, monoclonal immunoglobulin and receptor ligands have been selected as molecular platforms to facilitate selective “targeted” anthracycline delivery at membrane antigens or membrane receptor complexes highly over-expressed on the exterior surface of cells including the neoplastic cell types such as mammary adenocarcinoma, metastatic melanoma and multiple myeloma. Utilizing optimized semi-synthesis methodologies, anthracycline immunoconjugates have been produced that possess higher or relatively high (effective) levels of potency compared to molar-equivalent concentrations of the corresponding “free” chemotherapeutic. Interestingly, some doxorubicin immunochemotherapeutics that have low ex-vivo levels of potency exert surprisingly high levels of in-vivo anti-neoplastic potency.

Immunochemotherapeutics synthesized as anthracycline (C13-keto)-immunoglobulin with selectively “targeted” delivery capabilities for breast cancer have predominately utilized anti-Lewis Y antigen monoclonal antibody fractions (e.g. BR96/SGN15). In part this non-dedicated strategy has been applied because anti-Lewis Y antigen monoclonal immunoglobulin is also cross-effective for the relatively selective “targeted” delivery of chemotherapy against lung carcinoma, intestinal carcinoma, and ovarian carcinoma.

Gemcitabine has been covalently bound to a relatively small array of biologically relevant ligands such as poly-L-glutamic acid (polypeptide configuration), cardiolipin, 1-dodecylthio-2-decyloxypropyl-3-phophatidic acid, lipid-nucleosides, N-(2-hydroxypropyl)methacrylamide polymer (HPMA), benzodiazepine receptor ligand, 4-(N)-valeroyl, 4-(N)-lauroyl, 4-(N)-stearoyl, 1,1′,2-tris-nor-aqualenecarboxylic acid, and the 4-fluoro[¹⁸F]-benzaldehyde derivative for application as a positron-emitting radionuclide. When necessary, the C₄—NH₂, C₃′—OH and C₅′—OH groups of gemcitabine can be reversibly protected utilizing di-tert-dibutyl dicarbonate. Few if any reports have described the molecular design and efficacy evaluation of covalent gemcitabine immunochemotherapeutics synthesized that entail the generation of a covalent bond between either at the C₅-methylhydroxy or cytosine-like C₄-amine groups of gemcitabine.

### Attributes and Efficacy

Anthracyclines in their clinical application are among the most potent and effective class of chemotherapeutic currently utilized for the treatment of an array of carcinomas, acute myeloid leukemia, and many other neoplastic disease states. The most frequent sequelae associated with anthracycline administration is cardiotoxicity (doxorubicin>>epirubicin) and nephritic syndrome.

A common therapeutic deficiency of anti-HER2/neu monoclonal immunoglobulin (trastuzumab, pertuzumab) and other related immunoglobulin biologicals is their tendency to exert primarily cytostatic rather than cytocidal activity in HER2/neu over-expressing mammary carcinoma. Anti-EGFR monoclonal immunoglobulin (cetuximab) blocks the extracellular ligand-binding domain and inhibits signal transduction and associated tyrosine kinase activity. A universal lethal cytotoxic effect is not induced by anti-HER2/neu or anti-EGFR monoclonal immunoglobulins, and under conditions of high anti-HER2 pressure, refractory subpopulations become established that display slower proliferation rates, resistant to apoptotic-transformation, and G₁-arrest. Termination of anti-HER2/neu monoclonal immunoglobulin is complicated by regained HER2/neu over-expression and chemotherapeutic resistance that coincides with a reversal of neoplastic growth inhibition and a return to original “baseline” proliferation rate levels. Such cancer cell behaviors directly correlate with the observation that most conditions of metastatic breast cancer that over-express HER2/neu and initially respond to single-agent HER2/neu monoclonal antibody (Tratuzumab) develop progression of disease within 1 year, and develop a 66-88% rate of primary resistance, often manifested as brain metasteses. The high frequency of developing a state of refractoriness severely limits complete resolution of neoplastic disease unless anti-HER2 immunotherapy is combined with an anthracycline, or other chemotherapeutic agent. In this context, the combination of trastuzumab/anthracycline is more efficacious than trastuzumab/paclitaxel.

Collectively, covalent anthracyline-immunochemotherapeutics have been synthesized that have high levels of in-vitro cytotoxic anti-neoplastic potency against chemotherapeutic-resistant mammary carcinoma, CD38 positive MC/CAR multiple myeloma, B-lymphoma, melanoma, gastric carcinoma, colon carcinoma, and pulmonary carcinoma. In direct accord with their in-vitro level of cytotoxic anti-neoplastic potency, similar covalent anthracycline immunochemotherapeutics can reduce in-vivo tumor burden and prolong survival in human xenograft models for gastric carcinoma, breast cancer, CD38 positive MC/CAR multiple myeloma, B-lymphoma, T-cell lymphoma, colon carcinoma, ovarian carcinoma, pulmonary carcinoma, metastatic melanoma, hepatocellular carcinoma, and intracerebral small-cell lung carcinoma. Additionally, a number of clinical trials involved in evaluating the efficacy and potency of anthracycline-immunoconjugates continue to be conducted relevant to a small array of neoplastic conditions. A number of clinical trials have additionally been initiated to evaluate the efficacy and potency of anthracycline-immunoconjugates relevant to a small array of neoplastic disease states. In contrast to immunoglobulin-based diagnostic radiopharmaceuticals and radioimmunotherapeutics, there have been relatively few investigations conducted to date devoted to the design, synthesis and efficacy evaluation of anthracycline-immunoconjugates with selective anti-neoplastic properties against mammary carcinoma cell types propagated in-vitro in tissue culture, or implanted in-vivo as xenografts, or in clinical in-vivo efficacy trials.

The advantages of modalities that facilitate selective, targeted delivery of chemotherapeutics include; lower total dose requirements; potential opportunity to exert synergistic levels of cytotoxic anti-neoplastic activity (e.g. anti-HER2/neu in combination with a chemotherapeutic agent); reduced risk and frequency of side effects due to decreased chemotherapy exposure by innocent tissues/organ systems (e.g. cardiotoxicity, nephritic syndrome); viable strategy for circumventing complications related to common forms of chemotherapeutic resistance; higher localized deposition of chemotherapy; and improved over-all tolerance of chemotherapy. In contrast to immunoglobulin-based diagnostic radiopharmaceuticals and radioimmunotherapeutics, there have been relatively few investigations conducted to date that have been devoted to the molecular design, synthesis, and efficacy evaluation of anthracycline-immunoconjugates with selective anti-neoplastic properties against mammary carcinoma cell types propagated in-vitro in tissue culture, or as in-vivo xenografts, or in clinical in-vivo efficacy trials.

Gemcitabine is a deoxycytidine nucleotide analog that functions as a chemotherapeutic when intracellularly it becomes triphosphoralated allowing it in turn to substitute for cytidine during DNA replication resulting in its incorporation into DNA strands and the inhibition of DNA polymerase activity. A second mechanism-of-action for gemcitabine involves its inhibition and inactivation of ribonucleotide reductase which ultimately promotes suppression of deoxyribonucleotide synthesis in concert with diminished DNA repair and replication. Collectively these multiple mechanisms-of-action ultimately induce cellular apoptosis events. In clinical oncology, gemcitabine is administered for the treatment of certain leukemias and potentially lymphoma conditions in addition to a spectrum of adenocarcinomas and carcinomas affecting the lung (e.g. non-small cell), pancrease, bladder and esophogus. Gemcitabine has a brief plasma half-life because it is rapidly deaminated and the inactive metabolite is excreted into the urine. The molecular design and synthesis of covalent gemcitabine immunochemotherapeutics provides several attributes due to their ability to facilitate selective “targeted” chemotherapeutic delivery. In this molecular form, gemcitabine apparently becomes a poor substrate for both MDR-1 (multi-drug resistance efflux pump) and presumably the two rapid deaminating enzymes cytidine deaminase, and (following gemcitabine phosphorylation) and deoxycytidylate deaminase.

The covalent bonding of gemcitabine to trophic receptors like HER2/neu and EGFR that are frequently over-expressed in breast cancer and other carcinomas or adenocarcinomas allows an opportunity for achieving additive or synergistic levels of cytotoxic anti-neoplastic potency. Potential implications of this consideration pertain to the clinical efficacy of monoclonal immunoglobulin fractions with binding-avidity for HER2/neu and EGFR receptors have demonstrated effectiveness in the treatment of neoplastic conditions including mammary carcinoma/adenocarcinoma that highly over-express these trophic membrane receptors. Unfortunately, therapeutic monoclonal immunoglobulin fractions including anti-HER2/neu and anti-EGFR reportedly have an inability to exert significant cytotoxic activity or completely resolve neoplastic disease states unless they are applied in concert with chemotherapy or other forms of anti-cancer treatment.

Despite general familiarity with the influence of anti-HER2/neu immunoglobulin on the biology of cancer cells and its application in clinical oncology surprisingly little known about covalent gemcitabine-(anti-HER2/neu) immunochemotherapeutics and their potential to exert selectively “targeted” cytotoxic anti-neoplastic activity against chemotherapeutic-resistant breast cancer.

The present invention provides novel methods for the molecular design and the chemical synthesis of analogs having multiple mechanisms of action.

### SUMMARY OF THE INVENTION

The present invention provides for the molecular design and chemical synthesis of pharmaceutical-ligand analogs, pharmaceutical-pharmaceutical analogs, and similar molecular-molecular analogs with multiple mechanisms of action. Multi-phase and single-phase chemical reaction schemes have been developed for the synthesis of such analogs.

### EMBODIMENT-1

A multi-phase organic chemistry reaction scheme has been designed that employs the application of covalent bond-forming reagents that contain both an amine-reactive N-hydroxysuccinimide ester and a UV light activated diazirine (e.g. azipentanoate) group for the synthetic production of covalent pharmaceutical-receptor ligand analogs, pharmaceutical-immunoglobulin analogs, or pharmaceutical-synthetic ligand analogs, pharmaceutical-pharmaceutical analogs, and biologically active molecule-molecule analogs. The synthesis scheme that applies the multi-phase organic chemistry reactions has the advantages of employing relatively gentle reaction conditions, provides higher end-product yield, affords relatively rapid reaction times to completion, and lower reagent costs.

Utilizing the multi-phase organic chemistry reaction scheme, covalent pharmaceutical-ligand analogs have been designed, synthesized and evaluated for efficacy/potency. Examples include, but are not restricted to, covalent epirubicin (anthracycline) and gemcitabine immunochemotherapeutics that possess selective “targeted” delivery properties for specific neoplastic cell types or populations relevant to diseases commonly treated in clinical oncology (e.g. chemotherapeutic resistant mammary adenocarcinoma as a model for other malignant conditions). Additive and synergistic levels of cytotoxic anti-neoplastic potency are possible to achieve through the combined properties of the chemotherapeutic moiety and monoclonal immunoglobulin fractions (e.g. anti-HER2/neu, anti-EGFR, anti-VEGR, anti-IGFR) that possess, for example, inhibitory properties against trophic receptor complexes frequently over-expressed by several different neoplastic cell types (e.g. carcinomas).

Utilizing the multi-phase organic chemistry reaction scheme, small molecular weight molecule-molecule or pharmaceutical-pharmaceutical analogs have been designed and synthesized. The covalent pharmaceutical-pharmaceutical analogs that have been designed and synthesized include, but are not restricted to, chemotherapeutic-chemotherapeutic preparations that possess new/unique and/or multiple mechanisms of action such as cytotoxic anti-neoplastic potency/efficacy against a spectrum of neoplastic disease states.

Representative examples of pharmaceutical agents utilized for the synthesis of covalent pharmaceutical-ligand and pharmaceutical-pharmaceutical analogs include the anthracyclines, gemcitabine, eribulin, cytosine arabinoside (ara-C, cytarabine), 6-thioguanine, fludarabine (Fludara), 5-azacytidine, decitabine, lenalidomide, and temozolomide.

### EMBODIMENT-2

A multi-phase organic chemistry reaction scheme has been designed that employs the application of covalent bond-forming reagents that contain both an amine-reactive chemical group (e.g. N-hydroxysuccinimide ester) and a phosphate-reactive chemical group (e.g. carbodiimide analog) for the covalent bonding of biologically active molecules (e.g. pharmaceuticals, chemotherapeutics) to large molecular weight platforms including immunoglobulin, Fab′ F(ab′)₂, receptor ligands (e.g. EGF, VEGF, IGF), and receptor ligand fragments that can provide various biological properties including selective “targeted” delivery, progressive deposition, and intracellular accumulation. The synthesis scheme that applies the multi-phase organic chemistry reaction has the advantage of employing relatively gentle reaction conditions, provides higher end-product yields, affords relatively rapid reaction times to completion, and lower reagent/production costs.

Utilizing the multi-phase organic chemistry reaction scheme, covalent pharmaceutical-ligand analogs have been designed for the synthesis of fludarabine-immunochemotherapeutics, dexamethazone-immunotherapeutics and clindamycin-immunochemotherapeutics that possess selective “targeted” delivery properties for specific neoplastic cell types (populations) relevant to diseases commonly treated in clinical oncology. Clindamycin-immunochemotherapeutics have the potential of serve as a means for improved therapy of protozoal disease states (e.g. malaria). Additive and synergistic levels of cytotoxic anti-neoplastic potency is possible through the combined properties of the chemotherapeutic moiety and monoclonal immunoglobulin fractions (e.g. anti-HER2/neu, anti-EGFR, anti-VEGR) that, for example, possess inhibitory properties against trophic receptor complexes frequently over-expressed by neoplastic cell types (e.g. carcinomas). Similar concepts apply to the in-vivo administration of clindamycin-immunochemotherapeutics.

### EMBODIMENT-3

A single-phase organic chemistry reaction scheme has been designed that employs the application of covalent bond-forming reagents that contain two chemically reactive sites that form covalent bonds at identical chemical groups. A relevant example includes molecular reagent analogs that contain two amine-reactive N-hydroxysuccinimide esters groups (e.g. disuccinimidyl glutarate and similar analogs). Such molecular reagents can be applied for the chemical synthesis of molecular entities that possess new/unique and/or multiple mechanisms of action. Molecular moieties contain at least (preferably) one amine, or hydroxyl, or carboxyl, or phosphate chemical group that are used in combination with a reagent that contains identical chemically reactive groups that will form a covalent bond at the corresponding chemical group of a precursor pharmaceutical or precursor molecule that possesses or will ultimately evoke chemical properties or biological activity of therapeutic benefit. A single or dual combination of biologically active molecular moieties (e.g. pharmaceutical agent) can be applied during the synthesis procedure. Examples of pharmaceutical agents utilized include anthracyclines, gemcitabine, eribulin, cytosine arabinoside (aura-C, cytarabine), 6-thioguanine, fludarabine, 5-azacytidine, decitabine, lenalidomide, and temozolomide.

Related covalent bond forming reagents include, but are not restricted to, bis-[2-(succinimidooxycarbonyloxy)ethyl]sulfone) disuccinimidyl suberate, bis[sulfosuccinimidyl] suberate, dithiobis[succinimidyl propionate, ethylene glycol bis[sulfosuccinimidylsuccinate and ethylene glycol bis[succinimidylsuccinate. In a similar context, tris-succinimidyl aminotriacetate would provide the option of being able to synthesize a molecular complex that contains three identical pharmaceutical precursors, or two identical precursors in combination with a different third pharmaceutical precursor, or a molecular complex that contain three different pharmaceutical precursors. The covalent bond forming reagents in this class have the reported advantage of greater stability that the application of imidoesters as covalent bond forming reagents. Conceptually, a similar synthesis approach can employ covalent bond forming reagents that contain two sulfhydryl reacting groups (e.g. bismaleimidohexan; 1.4-bismaleimidyl-2-3-dihydroxybutane) which can be used to form a covalent pharmaceutical-pharmaceutical complex that utilizes either one or two pharmaceutical precursors that possess an available sulfhydryl group (e.g. 6-thioguanine) as seen in the illustration provided (FIG. 4B).

### EMBODIMENT-4

A single-phase or multi-phase organic chemistry reaction scheme has been designed that employs the application of covalent bond-forming reagents that contain two different chemically reactive sites or groups which create a covalent bond with two different pharmaceuticals that each possess a different (unique) type of chemical group. Examples of chemically reactive moieties of covalent bond forming reagents includes, N-hydroxysuccinimide ester analogs (amine-reactive), isocyanate analogs (hydroxyl reactive), carbodiimide analogs (phosphate-reactive and carboxyl-reactive), hydrazide analogs (carbonyl-reactive), and maleimides (sulfhydryl reactive). Such molecular bonding forming reagents can be applied in synthetic organic chemistry reactions to produce molecular entities that possess new/unique and/or multiple mechanisms-of-action. Ideally, each pharmaceutical agent (or other molecule that contains chemical properties or biological activity) must contain at least (preferably) one amine, or one hydroxyl, or one sulfhydryl, or one carboxyl, or one phosphate or one carbonyl chemical group. In practice, two different precursor pharmaceuticals (e.g. or other molecule with chemical properties or biologically activity) are applied that each contain a single chemical group that can react and form a covalent bond with one of the two chemically reactive groups of the covalent bond forming reagent. The term pharmaceutical in this context is used in broad terms to include pharmaceuticals, chemotherapeutics, nucleotide sequences (e.g. siRNA) and other molecules that possess or can evoke biological activity or chemical properties.

With the foregoing and other objects, features, and advantages of the present invention that will become apparent hereinafter, the nature of the invention may be more clearly understood by reference to the following detailed description of the preferred embodiments of the invention and to the appended claims.

### BRIEF DESCRIPTION OF THE DRAWINGS

These drawings accompany the detailed description of the invention and are intended to illustrate further the invention and its advantages:

FIG. 1 depicts a schematic illustration of the chemical reactions utilized for the synthesis of epirubicin-(C₃-amide)[anti-HER2/neu]. Legend: (Phase-I) creation of a covalent amide bond at the C₃ monoamine of epirubicin and the ester group of succinimidyl 4,4-azipentanoate resulting in the creation of a covalent UV-photoactivated epirubicin-(C₃-amide) intermediate accompanied by the liberation of the succinimide “leaving” complex; (Phase-II) creation of a covalent bond between the UV-photoactivated epirubicin-(C₃-amide) intermediate and amino acid residues within the sequence of anti-HER2/neu monoclonal immunoglobulin initiated by photoactivation (UV 354 nm).

FIG. 2 depicts a schematic illustration of the organic chemistry reactions utilizing a 2-phase synthesis scheme for gemcitabine-(C₄-amide)-[anti-HER2/neu]. Legends Plate 2A: (Phase-I) creation of a covalent amide bond at the C₄ monoamine of gemcitabine and the ester group of succinimidyl 4,4-azipentanoate resulting in the creation of a covalent UV-photoactivated gemcitabine-(C₄-amide) intermediate accompanied by the liberation of the succinimide “leaving” complex. (Phase-II) creation of a covalent bond between the UV-photoactivated gemcitabine-(C₄-amide) intermediate and amino acid residues within the amino acid sequence of anti-HER2/neu monoclonal immunoglobulin initiated by photoactivation (UV 354 nm). Legends Plate 2B: creation of an amide bond at the C₄ monoamine group of gemcitabine and the ester group of succinimidyl 4,4-azipentanoate resulting in the creation of a covalent UV-photoactivated gemcitabine-(C₄-amide) intermediate accompanied by the liberation of the succinimide “leaving” complex. (Phase-II) creation of a covalent bond between the UV-photoactivated gemcitabine-(C₄-amide) intermediate and a chemical group of a second molecule of gemcitabine chemotherapeutic initiated by photoactivation (UV 354 nm).

FIG. 3 depicts a schematic illustration of the organic chemistry reactions utilizing a 2-phase synthesis scheme utilized to covalently bond a phosphate (—PO₄) pharmaceutical analog to a large molecular weight platform (e.g. immunoglobulin, receptor ligand) at the ε-amine (—NH₂) group of lysine residues within their amino acid sequence. Large molecular weight platforms can include protein fractions (e.g. monoclonal immunoglobulin, Fab′, receptor ligands, receptor ligand fragments) or synthetic preparations.

FIG. 4 depicts a schematic illustration of the organic chemistry reactions utilizing a 1-phase synthesis scheme utilized for the synthesis of covalent small molecular weight complexes. Legends: (Plate 4a) creation of covalent amide bonds at the C₄ monoamine of two gemcitabine molecules and the two ester groups of disuccinimidyl glutarate. Legends: (Plate 4B) creation of covalent amide bonds at the sulfhydryl (—SH) group of two 6-thioguanine molecules and the two maleimide groups of bis-(malimeido)-ethane.

FIG. 5 depicts a schematic illustration of the organic chemistry reactions utilizing a 1-phase synthesis scheme utilized for the synthesis of covalent small molecular weight complexes. Legends: (Plate 5a) creation of a covalent amide bond at the monoamine groups of both gemcitabine and clidarabine with the two ester groups of disuccinimidyl glutarate. (Plate 5b) creation of a covalent bond at the extended chain primary hydroxyl (—OH) of gemcitabine and primary ring sulfhydryl (—SH) of 6-thioguanine utilizing p-maleimidophenyl isocyanate as a covalent bond-forming reagent; (Plate 5c) generation of a covalent bonds between the phosphate (—PO₄) group of fludarabine phosphate, and the amine (—NH₂) group of gemcitabine.

FIG. 6 depicts the analysis of end-product reactants by high-performance thin-layer chromatography (HP-TLC) applying the covalent bond-forming reagent disuccinimidyl glutarate in combination with either cytosine arabinoside (Ara-C) or decitabine chemotherapeutics. Legends: (Lane-1) cytosine arabinoside reference control; (Lane 2) N-hydroxysuccinate reference control; (Lanes 3, 4 & 5) chemotherapeutics with a cyclic primary amine reacted with disuccinimidyl covalent bond forming reagent at a 7.5:1 molar excess in DMSO supplemented with TEA (50 mM final concentrations). Arrow indicates the production of the desired end-product based upon the combined interpretation of Rf value and mass-spectrometry analyses. Development of silica-Gel HP-TLC plates was performed utilizing a propanol/ethanol/H₂O solvent phase with identification established by UV elimination.

### DETAILED DESCRIPTION OF THE INVENTION

Synthesis of Covalent Pharmaceutical-Ligands or Immunochemotherapeutics: Method 1

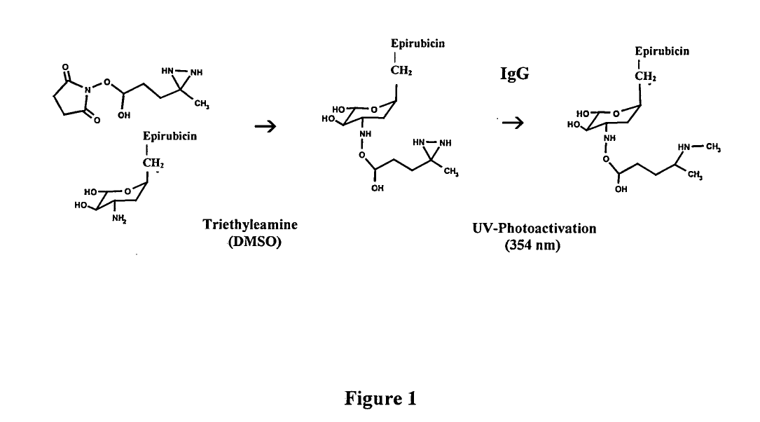

Phase-I Synthesis Scheme for UV-Photoactivated Pharmaceutical Intermediates-The (primary) amine group of a pharmaceutical (e.g. 2.80×10⁻³ mmoles) is reacted at a 2.5:1 molar-ratio with the amine-reactive N-hydroxysuccinimide ester “leaving” complex (e.g. succinimidyl 4,4-azipentanoate (0.252 mg, 1.12×10⁻³ mmoles) in the presence of triethylamine (50 mM final concentration) utilizing dimethylsulfoxide as an anhydrous organic solvent system (FIGS. 1 & 2A). The reaction mixture formulated from stock solutions of epirubicin and succinimidyl 4,4-azipentanoate is then continually stirred gently at 25° C. over a 4-hour incubation period in the dark and protected from exposure to light. The relatively long incubation period of 4 hours is utilized to maximize degradation of the ester group of any residual succinimidyl 4,4-azipentanoate that may not of reacted in the first 30 to 60 minutes with the pharmaceutical amine group. Mono-amine pharmaceuticals are the preferred agents for Phase-I synthesis procedures.

Phase-II Synthesis Scheme for Covalent Pharmaceutical-Ligands Utilizing a UV-Photoactivated Pharmaceutical Intermediate-Molecular ligand platforms that contain primary amine groups (e.g. monoclonal immunoglobulin fractions; 1.5 mg, 1.0×10⁻⁵ mmoles) in buffer (PBS: phosphate 0.1, NaCl 0.15 M, EDTA 10 mM, pH 7.3) are combined at a 1:10 molar-ratio with the UV-photoactivated pharmaceutical intermediate (Phase-I end product) and allowed to gently mix by constant stirring for 5 minutes at 25° C. in the dark. The photoactivated group of the pharmaceutical-intermediate then forms a covalent bond with amino acid residues within the sequence of peptides or proteins (e.g. monoclonal immunoglobulin, receptor fragments) during a 15 minute exposure to UV light at 354 nm (reagent activation range 320-370 nm) in combination with constant gentle stirring (FIGS. 1 & 2). Residual pharmaceutical is removed from covalent pharmaceutical-ligand preparations applying micro-scale “desalting” column chromatography with the media pre-equilibrated with PBS (phosphate 0.1, NaCl 0.15 M, pH 7.3).

### Synthesis of Covalent Pharmaceutical-Ligands or Immunochemotherapeutics: Method 2

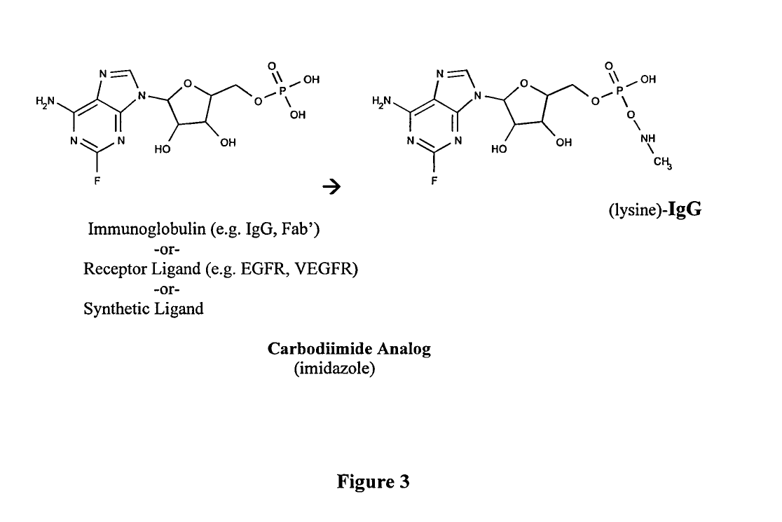

Phase-I Synthesis Scheme for a Phosphate-Reactive Pharmaceutical Intermediate-The (primary) phosphate group of a pharmaceutical agent (e.g. 2.80×10⁻³ mmoles) is reacted at a 10:1 to 2:1 molar-ratio with the phosphate-reactive group (e.g. carbodiimide analog in combination with imidazole) of a covalent bond forming agent (e.g. 1-ethyl-3[3-dimethylaminopropyl]-carbodiimide). In a non-phosphate buffer system, the reaction mixture formulated from stock solutions is then continually stirred gently at 25° C. over a 15 minute to 4-hour incubation period. Mono-phosphate forms of pharmaceutical are the preferred agents for Phase-I synthesis procedures (FIG. 3).

Phase-II Synthesis Scheme for Covalent Pharmaceutical-Ligands Utilizing an Amine-Reactive Pharmaceutical Intermediate-Molecular ligand platforms that contain primary amine groups (e.g. monoclonal immunoglobulin fractions; 1.5 mg, 1.0×10⁻⁵ mmoles) in an aqueous buffer (HEPES or carbonate 0.1, NaCl 0.15 M, EDTA 10 mM, pH 7.3) are combined at a 1:10 molar-ratio with the amine-reactive pharmaceutical intermediate (Phase-I end product) and allowed to gently mix by constant stirring at 25° C. for 15 minutes to 4 hours. The subsequent synthetic organic chemistry reaction results in the pharmaceutical-intermediate forming a covalent bond at the ε-amine group of lysine residues with the amino acid sequence of peptides or proteins that can include monoclonal immunoglobulin, immunoglobulin fragments (e.g. Fab′, F(ab′)₂ receptor ligands (e.g. EGFR, VEGFR), or receptor fragments (FIG. 3). Residual un-reacted pharmaceutical is removed from covalent pharmaceutical-ligand preparations applying micro-scale “desalting” column chromatography with the media pre-equilibrated with PBS (phosphate 0.1, NaCl 0.15 M, pH 7.3).

### Synthesis of Covalent Pharmaceutical-Pharmaceutical Analogs Utilizing a UV-Photoactivated Intermediate: Method 3

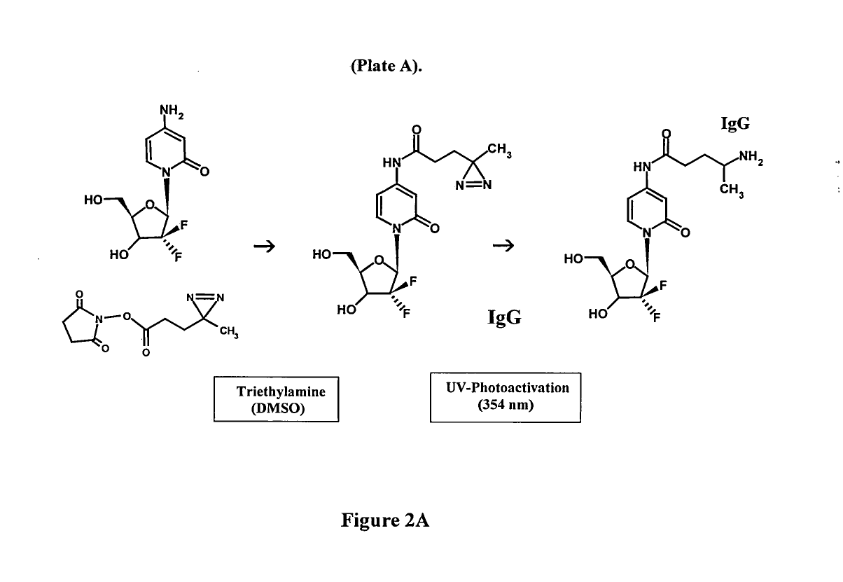

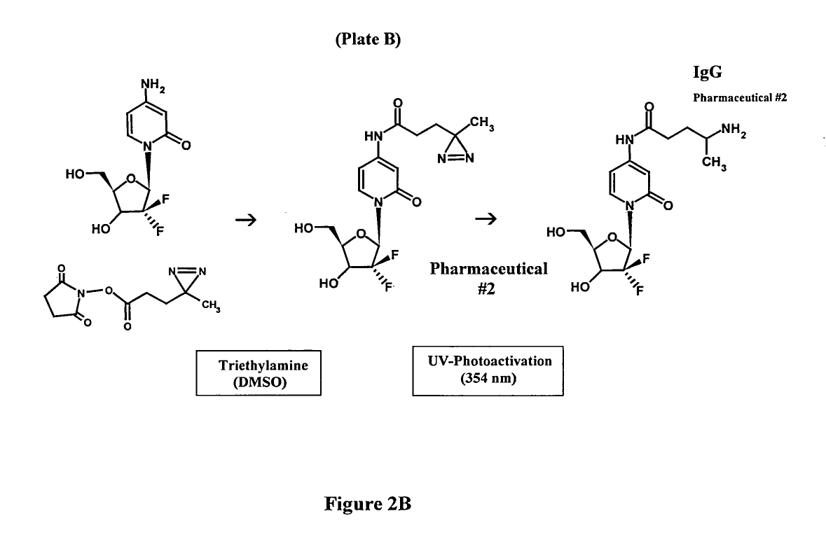

Phase-I Synthesis Scheme for UV Pharmaceutical Pharmaceutical Intermediates-The (primary) amine group of a pharmaceutical (e.g. 2.80×10⁻³ mmoles) is reacted at a 1:2.5 molar-ratio with the amine-reactive N-hydroxysuccinimide ester “leaving” complex (e.g. succinimidyl 4,4-azipentanoate (0.252 mg, 1.12×10⁻³ mmoles) in the presence of triethylamine (50 mM final concentration) utilizing dimethylsulfoxide as an anhydrous organic solvent system (FIG. 2 Plate B). The reaction mixture formulated from stock solutions of epirubicin and succinimidyl 4,4-azipentanoate is then continually stirred gently at 25° C. over a 4-hour incubation period in the dark and protected from exposure to light. The relatively long incubation period of 4 hours is utilized to maximize degradation of the ester group of any residual succinimidyl 4,4-azipentanoate that may not of reacted in the first 30 to 60 minutes with the pharmaceutical amine group. Mono-amine pharmaceutical are the preferred agents for synthesis procedures.

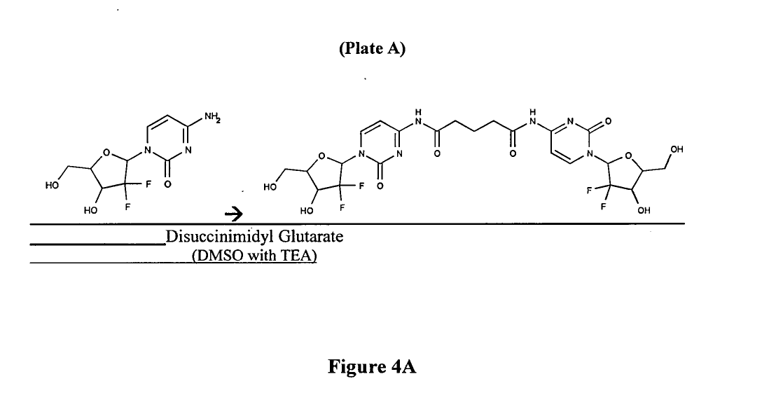

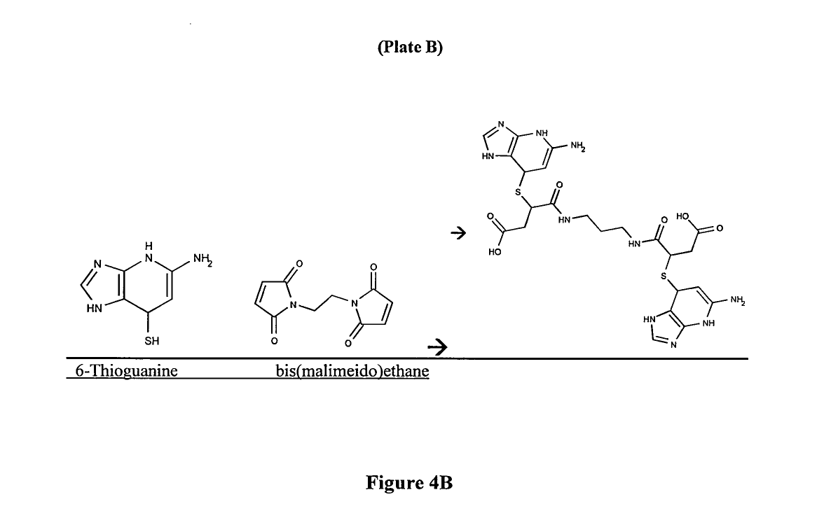

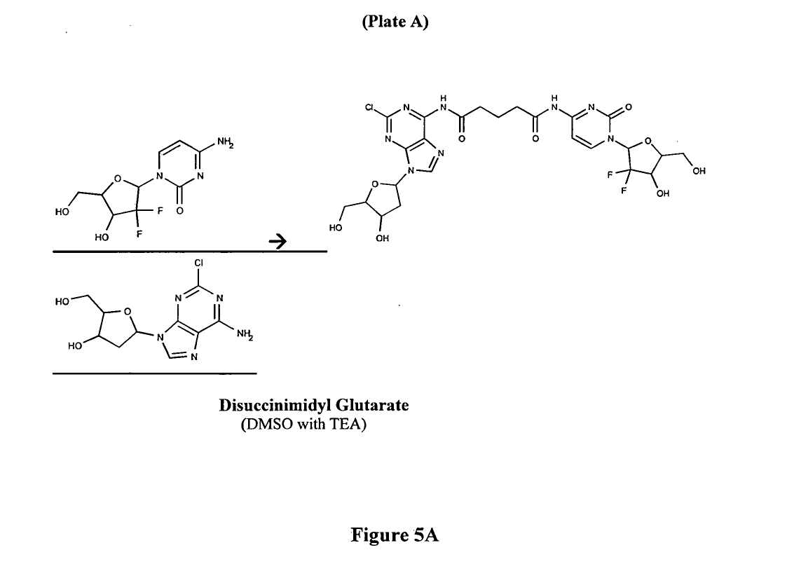

Phase-II Synthesis Scheme for Covalent Pharmaceutical-Pharmaceutical Analogs Utilizing a UV-Photoactivated Pharmaceutical Intermediate-A second pharmaceutical that contains primary amine group (e.g. 2.8×10⁻³ mmoles in dimethylsulfoxide) is combined with the UV-photoactivated pharmaceutical intermediate (Phase-I end product) and allowed to gently mix by constant stirring for 5 minutes at 25° C. in the dark. The photoactivated group of the pharmaceutical-intermediate is then preferentially reacted with the primary amine group of the second pharmaceutical during a 15 minute exposure to UV light at 354 nm (reagent activation range 320-370 nm) in combination with constant gentle stirring (FIG. 4 Plate A). Purification of the final covalent pharmaceutical-pharmaceutical end-product is achieved utilizing preparative-scale high-performance thin layer chromatograph in combination with a propanol/chloroform/H₂O mobile phase solvent system (1:1:1).

Synthesis of Covalent Pharmaceutical-Pharmaceutical Analogs with Identical Moieties: Method 4

Phase-I Synthesis Scheme for Pharmaceuticals Using Primary Amine, Hydroxyl, Carboxyl, or Sulfhydryl, Phosphate, or Carbonyl Molecular Precursors-The chemical group of a single biologically active molecular moieties (e.g. pharmaceutical/pharmaceuticals) are combined at a 40:1 to 2:1 molar ratio with a covalent bond-forming reagent that contains two identical chemically reactive sites or groups utilizing an organic (dimethylsulfoxide DMSO; Dimethyl fluoride, DMF) or aqueous (e.g. water ddH₂O; phosphate buffered saline 0.9 M pH 7.4) solvent systems (FIG. 4 Plate A). The chemical groups of the covalent bond-forming reagent can react with amines (e.g. N-hydroxysuccinimide esters), hydroxyls (e.g. isocyanates), carboxyls (carbodiimide), sulfhydryls (e.g. maleimides), phosphates (carbodiimide analogs), or carbohydrates (hydrazides that react with carbonyl groups like aldehyde or ketones, either innately present or formed by gentle oxidation). Corresponding chemical groups on molecules where covalent bonds are formed include primary amines, hydroxyls, carboxyls, sulfhydryls phosphates, and carbonyls (e.g. aldehydes, ketones of oxidized carbohydrates). In organic solvent systems, triethylamine (TEA 50 mM final concentration) can be applied to enhance the progress of reactions in the absence of water if necessary.

A relevant synthesis example would include the application of a covalent bond forming agent that contains two N-hydroxysuccinimide ester “leaving” complexes (e.g. disuccinimidyl glutarate) in the presence of water (ddH₂O), phosphate buffered saline (0.9 M pH 7.4) or dimethylsulfoxide (DMSO with triethylamine 50 mM final concentration) (FIG. 4 Plate A). The covalent bond forming reagent is then combined with the molecule that will ultimately impart biological or chemical activity (e.g. pharmaceutical or dual pharmaceutical combination) and contains a primary amine group (FIG. 4, Plate A). The reaction mixture is then allowed to continually stir gently at 25° C. over an incubation period that can range from 4-to-200 hours. Relatively longer incubation periods can be employed for the synthesis of covalent bond structures at chemical groups associated with aeromatic ring structures. Subsequent separation or purification can be attained by either HP-TLC or HPLC in concert with mass spectrometry and NMR analysis.

An analogous approach includes a methodology that employs a covalent bond forming reagent that contains two sulfhydryl reacting groups (e.g. maleimides or maleimidos) that will react at the sulfhydryl groups (—SH) of pharmaceuticals or molecular agents that possess biological activity or exert chemical properties (FIG. 4 Plate B).

Synthesis of Covalent Pharmaceutical-Pharmaceutical Analogs with Different Moieties: Method 5

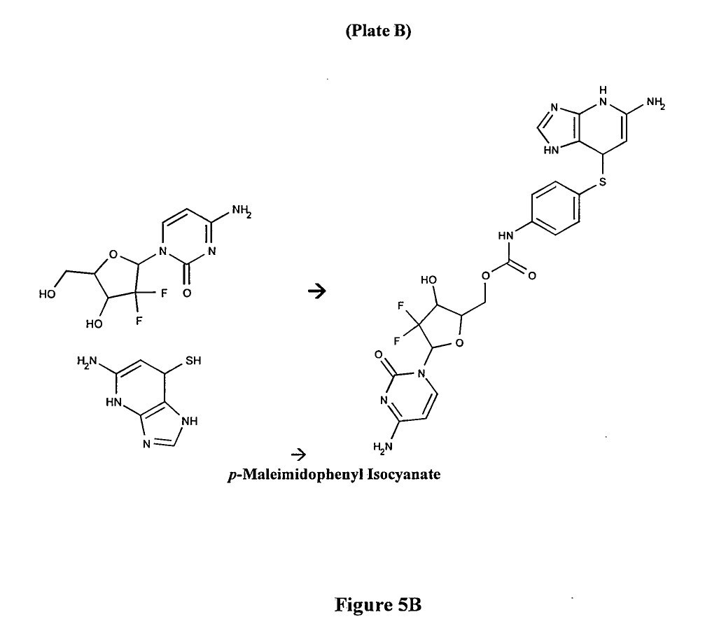

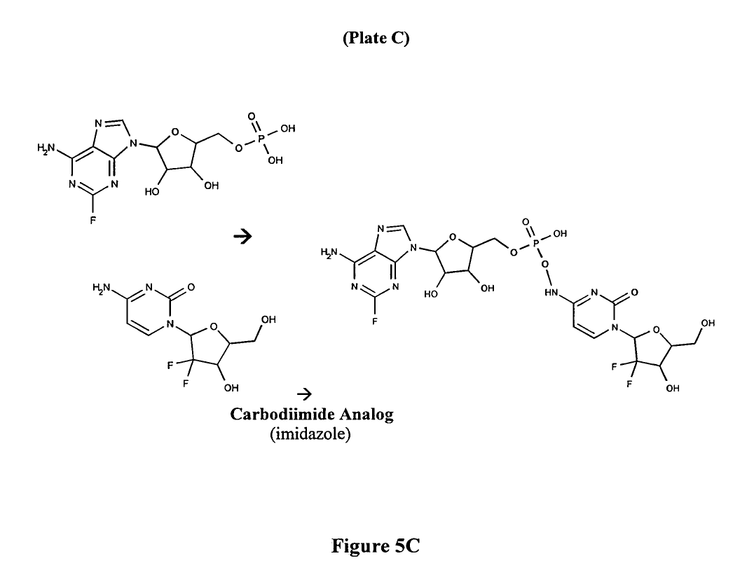

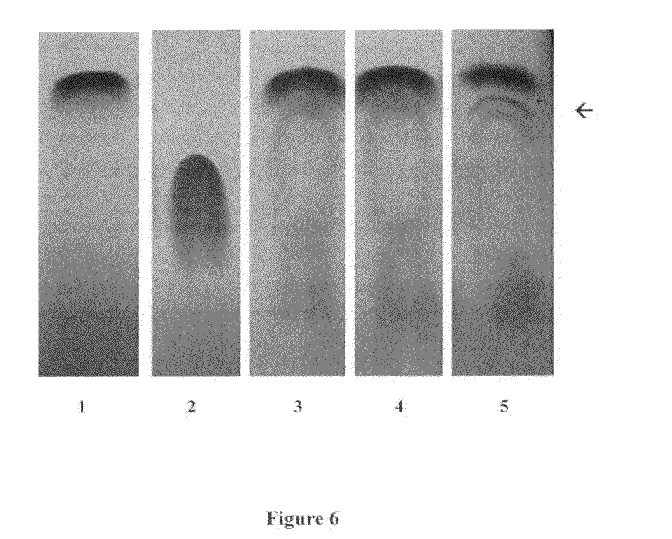

Phase-I Synthesis Scheme for Pharmaceuticals Using Primary Amine, Hydroxyl, Carboxyl, or Sulfhydryl, Phosphate, or Carbonyl Molecular Precursors-The chemical group of multiple different biologically active molecular moieties (e.g. pharmaceutical/pharmaceuticals) are combined at a 40:1 to 2:1 molar ratio with a covalent bond-forming reagent that contains two different chemically reactive sites or groups utilizing an organic (dimethylsulfoxide DMSO; Dimethyl fluoride, DMF) or aqueous (e.g. water ddH₂O; phosphate buffered saline 0.9 M pH 7.4) solvent systems (FIG. 5 Plates A, B & C). The chemical groups of the covalent bond-forming reagent can react with amines (e.g. N-hydroxysuccinimide esters), hydroxyls (e.g. isocyanates), carboxyls (carbodiimide), sulfhydryls (e.g. maleimides), phosphates (carbodiimide analogs), or carbohydrates (hydrazides that react with carbonyl groups like aldehyde or ketones, either innately present or formed by gentle oxidation). Corresponding chemical groups on molecules where covalent bonds are formed include primary amines, hydroxyls, carboxyls, sulfhydryls phosphates, and carbonyls (e.g. aldehydes, ketones of oxidized carbohydrates). In organic solvent systems, triethylamine (TEA 50 mM final concentration) can be applied to enhance the progress of reactions in the absence of water if necessary (FIG. 5 Plates A, B & C).

### Attributes and Implications

Molecular design and synthesis of gemcitabine-(C₄-amide)[anti-HER2/neu] utilizing a UV-photoactivated gemcitabine-(C₄-amide) intermediate created with the application of succinimidyl 4,4-azipentanoate represents a molecular design and organic chemistry scheme that has only once previously been applied for the production of epirubicin-(C₄-amide)-[anti-HER2/neu]. A somewhat unique property of the UV-photoactivated gemcitabine-(C₄-amide) intermediate generated utilizing succinimidyl 4,4-azipentanoate in Phase-I of the synthesis scheme is that it does not contain a sulfhydryl-reactive maleimide group (FIG. 1). Ultimately this allows the implementation of a reaction scheme for the synthesis of gemcitabine-(C₄-amide)-[anti-HER2/neu] that does not require the creation or introduction of reduced sulfhydryl groups into the amino acid sequence of whole immunoglobulin, F(ab′)₂, Fab or other biologically active proteins (FIG. 1). In contrast, the gemcitabine-(C₅ methylcarbamate) reactive intermediate synthesized with N[p-maleimidophenyl]-isocyanate does contain a sulfhydryl-reactive maleimide group (FIG. 2). Similarly, the anthracycline reactive intermediates applied during the synthesis of many if not most anthracycline-immunochemotherapeutics also employ a sulfhydryl-reactive maleimide group to facilitate the creation of a covalent bond with immunoglobulin or other biologically active protein fractions. Examples in this regard include synthesis schemes that are dependent upon heterobifunctional reactants similar to succinimidyl-4-(N-maleimidomethyl)cyclohexane-1-carboxylate (SMCC), N-ε-maleimidocaproic acid hydrazide (EMCH), or N-[p-maleimidophenyl]-isocyanate (PMPI). In the application of these reagents, pre-thiolation of immunoglobulin fractions and other biological proteins is usually required due to the relatively low number of non-sterically hindered sulfhydryl groups in the form of reduced cysteine amino acid residues (e.g. R—SH) available within the amino acid sequence of most biologically active proteins. Increasing the number of available reduced sulfhydryl groups can be achieved by the application of 1,4-dithiothreitol which reduces intramolecular cystine-cystine bonds and similar disulfide structures (DTT: R—CH₂—S—S—CH₂—R→2R—CH₂—SH). The actual synthetic introduction of “new” or additional reduced sulfhydryl groups at the ε-amine of lysine residues within the amino acid sequence is possible with reactions that utilize 2-iminothiolane (2-IT), mercaptosuccinimide, or N-succinimidyl-S-acetylthioacetate (SATA). Alternatively, carboxyl groups on molecules like heparin and hyaluronic acid (HA) can be thiolated with 3,3′dithiobis(propanoic)-hydrazide (DPTH) or divinylsulfone (DVS), and hydroxyl groups of molecules with a cholesterol-like core. In the application of DTPH, the integral disulfide bond is subsequently reduced with DTT reagent.

Due to a lack of a sulfhydryl-reactive maleimide group in the gemcitabine-(C₄-amide) intermediate, and because it is instead almost solely reactive with a-amine groups under the conditions applied, there is in turn no requirement for the pre-thiolation of immunoglobulin,

F(ab′)₂, Fab or other biologically active proteins (FIG. 1). Covalently bonding gemcitabine or other chemotherapeutic agents to biological protein fractions like immunoglobulin without a requirement to convert existing cystine-cystine disulfide bonds to their reduced form or the synthetic introduction of reduced sulfhydryl groups is a distinct advantage. Such synthesis schemes entail the implementation of fewer synthetic chemistry reactions, require smaller amounts of critical reagents, and maximize final yield in part due to at least one less column chromatography separation procedure. The brief duration of the synthesis scheme for gemcitabine-(C₄-amide)[anti-HER2/neu] utilizing succinimidyl 4,4-azipentanoate is also possible because of the relatively rapid time course for Phase-I and especially the Phase-II organic chemistry reactions, and because the methodology has been designed so that adjustment of buffer pH to different levels during the procedure is not necessary in contrast to other techniques.

Perhaps one of the most important features of the synthesis methodology is a lack of a requirement for cystine-cystine disulfide bond reduction or pre-thiolation allows by design the application of synthetic chemistry reactions that are highly efficient under relatively milder conditions that promote a lower risk of protein fragmentation or secondary polymerization through premature inter-molecular and intra-molecular disulfide bond formation. Realized benefits therefore include greater retained biological activity (e.g. antigen binding-avidity) and total final yield. Lastly, lack of a requirement for either converting existing cystine-cystine disulfide bonds to their reduced form or the introduction of reduced sulfhydryl groups into immunoglobulin fractions reduces restrictions and limitations on the magnitude of the molar-incorporation-index that can be attained. In contrast, the chemotherapeutic incorporation index for covalent immunochemotherapeutics synthesized utilizing SMCC, EMCH or PMPI is limited or restricted to levels only as high or lower than the extent of lysine ε-amine pre-thiolation. In pre-thiolation dependent synthesis schemes, higher epirubicin molar-incorporation-indexes are possible with modifications in methodology but the resulting harsher synthesis conditions are accompanied by substantial reductions in total yield of covalent immunochemotherapeutic, and declines in antigen-immunoglobulin binding-avidity (e.g. cell-ELISA parameters).

Implementation of succinimidyl 4,4-azipentanoate in the synthesis scheme for gemcitabine-(C₄-amide)-[anti-HER1/neu] has other desirable attributes besides a lack of a requirement for the pre-thiolation of immunoglobulin or other selective “targeted” delivery platforms that possess biological activity. In contrast to SMCC, EMCH or PMPI the synthesis of gemcitabine-(C₄-amide)-[anti-HER2/neu] utilizing succinimidyl 4,4-azipentanoate has the added benefit of not introducing biologically irrelevant five and six carbon or carbon/nitrogen ring structures into the final form of covalent immunochemotherapeutics (FIGS. 1 & 2). Elimination of any extraneous ring structures decreases the probability of inducing an in-vivo humoral immune response when administered by IV injection that can ultimate result in the formation of neutralizing antibody and increased risk of a post-treatment immune hypersensitivity reaction. The Phase-I reaction can be performed either in an aqueous buffer system or in an organic solvent system containing a low concentration of triethylamine [N(CH₂CH₃)₃] or other proton acceptor molecule. In stock solutions or reaction mixtures that contain an aqueous buffer solution significant hydrolytic degradation of succinimidyl 4,4-azipentanoate occurs. Alternatively, if stock solutions and reaction mixtures of epirubicin with succinimidyl 4,4-azipentanoate are instead formulated in an anhydrous organic solvent like DMSO in combination with a proton acceptor molecule then the resulting UV-photoactivated gemcitabine-(C₄-amide) intermediate is stable at 4° C. or −20° C. for a period of time when adequately protected from UV-light exposure. Such properties for succinimidyl 4,4-azipentanoate further demonstrate the convenient options of the synthesis method described that are in part facilitated by the ability to “pre-synthesize” and store the UV-photoactivated gemcitabine-(C₃-amide) intermediate for an extend period of time for the future production of a covalent gemcitabine-immunochemotherapeutic. The design of the synthesis scheme described offers still another added level of convenience because it illustrates a model method that can be adapted and modified to facilitate the covalent bonding of an array of different chemotherapeutic agents to a wide range of immunoglobulins (e.g. IgG, Fab′), receptor ligands or similar biologically active protein fractions.

### Preferred Embodiments

A single or multi-phase organic chemistry reaction scheme that employs the application of covalent bond-forming reagents that contain two chemically reactive groups that form covalent bonds at corresponding chemical groups within the structure of molecular entities that possess or will in their final form evoke chemical properties or biological activity (e.g. pharmaceuticals, nucleotides like siRNA sequences). Covalent bond-forming reagents can contain two of the same, or two different chemically reactive groups including for example those that are amine-reactive (e.g. N-hydroxysuccinimide ester analogs), hydroxyl-reactive (e.g. isocyanate analogs), phosphate-reactive (e.g. carbodiimide analogs), carboxyl-reactive (e.g. carbodiimide analogs), carbonyl-reactive (e.g. hydrazide analogs), and/or sulfhydryl-reactive (e.g. maleimide analogs). In addition to a wide spectrum of natural or synthestic biologically active molecules (e.g. siRNAs), a number of pharmaceutical agents can also be utilized as synthesis precursors including but not limited to anthracyclines, gemcitabine, eribulin, cytosine arabinoside (ara-C, cytarabine), 6-thioguanine, fludarabine (Fludara), 5-azacytidine, decitabine, lenalidomide, temozolomide, steroids (e.g. dexamethazone), and phosphated steroid analogs (e.g. dexamethazone phosphate). The natural or synthetic molecules or pharmaceutical agents that do or will possess chemical properties or biological activity each possess a chemical group that can react with the covalent bond forming reagent and include but is not limited to amine, hydroxyl, carboxyl, carbonyl, phosphate or sulfhydryl groups. Both aqueous (e.g. H₂O, carbonate, phosphate buffered saline) and organic (e.g. DMSO, DMF supplemented +/−triethylamine) solvent systems may be employed utilizing reagent molar rations, extended incubation periods and high reagent concentrations to maximize end-product yield.

A single or multi-phase organic chemistry scheme that employs the application of covalent bond-forming reagents that contain both a UV light activated diazirine (e.g. azipentanoate) group in addition to an amine-reactive group (e.g. N-hydroxysuccinimide ester analogs), or hydroxyl-reactive group (e.g. isocyanate analogs), or phosphate-reactive group (e.g. carbodiimide analogs), or carboxyl-reactive group (e.g. carbodiimide analogs), carbonyl-reactive group (e.g. hydrazide analogs), or sulfhydryl-reactive group (e.g. maleimide analogs). First, the amine-reactive (or hydroxyl-reactive, phosphate-reactive, carboxy-reactive, sulfhydryl-reactive) group of the covalent bond forming reagent is reacted with the corresponding amine, phosphate, carboxyl, carbonyl, or sulfhydryl group of a pharmaceutical thereby creating a covalent bond structure. The resulting UV-activated pharmaceutical intermediate is then covalently bound to large molecular weight platforms by exposure to UV-light of a specific wave-length range. Large molecular weight platforms can include but are not to be limited to immunoglobulin (e.g. anti-HER2/neu, anti-EGFR, anti-VEGFR, anti-IGFR), immunoglobulin fragments [e.g. Fab, F(ab₂)], receptor ligand fractions (e.g. EGF or EGF fragment, VEGF or VEGF fragment, IGF or IGF fragment), natural ligands (e.g. lectins, peptides, carbohydrates), synthetic ligand analogs (e.g. peptides, carbohydrates, aminated carbohydrates, partially oxidized carbohydrates, nucleotide sequences) or another pharmaceutical agent. The resulting end-product generated is in the form of a covalent pharmaceutical-receptor ligand, covalent pharmaceutical-immunoglobulin analog, pharmaceutical-immunoglobulin fragment analogs [e.g. Fab, F(ab₂)], pharmaceutical-synthetic ligand, or pharmaceutical-pharmaceutical analog.

The term pharmaceutical is broadly applied to include both molecules and pharmaceuticals that contain an available (e.g. primary) amine, or hydroxyl, or carboxyl, or carbonyl, or sulfhydryl group and possess or are capable of imparting chemical properties or biological activity of therapeutic benefit in their final form (e.g. pharmaceutical, chemotherapeutic, siRNA sequence, peptide, biochemically active enzyme). Pharmaceuticals or chemotherapeutics utilized as precursors in the organic chemistry reaction scheme can include but not be restricted to the anthracyclines, gemcitabine, eribulin, cytosine arabinoside (ara-C, cytarabine), 6-thioguanine, fludarabine (Fludara), 5-azacytidine, decitabine, lenalidomide, and temozolomide. Biologically or chemically active molecules and pharmaceuticals in the form of a chemically reactive intermediate are covalently bound to large molecular weight platforms that possess various biological properties including but not restricted to selective “targeted” delivery; prevention of rapid renale clearance (size-inhibited renal excretion and prolongation of plasma T_{1/2}); or modification of transport across biological barriers.

A multi-phase organic chemistry scheme that employs the application of covalent bond-forming reagents that contain both a phosphate-reactive group (e.g. carbodiimide analog supplemented with an imidazole analog) and an amine-reactive group (e.g. N-hydroxysuccinimide ester) for the synthetic production of covalent pharmaceutical-receptor ligand analogs (e.g. EGFR or EGFR fragment, VEGFR or VEGFR fragment, or IGF or IGF fragment), pharmaceutical-immunoglobulin analogs (e.g. IgG), pharmaceutical-immunoglobulin fragment analogs [(e.g. Fab, F(ab₂)], pharmaceutical-natural ligands (e.g. lectins, peptides, carbohydrates), pharmaceutical-synthetic ligands (e.g. peptides, carbohydrates, aminated charbohydrates, partially oxidized carbohydrates, nucleotide sequences like siRNA) and potentially pharmaceutical-pharmaceutical analogs. In the above examples, the term pharmaceutical is broadly applied to include both molecules and pharmaceuticals with an available phosphate group (e.g. —PO₄₋) that possess or are capable of imparting chemical properties or biological activity of therapeutic benefit (e.g. pharmaceuticals, chemotherapeutics, siRNA sequences, peptides, biochemically active enzymes). Phosphated pharmaceutical or chemotherapeutic precursors utilized in the organic chemistry reaction scheme can include but not be limited to phosphated steroid analogs (e.g. dexamethazone-phosphate, phosphated analogs of steroid hormone agonists or antagonists), phosphated chemotherapeutics (e.g. fludrabine/Fludara), and phosphated antibiotic/anti-protozoal agents (e.g. clindamycin phosphate). Biologically or chemically active molecules can be covalently bound to large molecular weight platforms that possess various biological properties including but not restricted to selective “targeted” delivery, prevention of rapid renale clearance (size-inhibited renal excretion and prolongation of plasma T_{1/2}) or modified transport across biological barriers.

Reaction end-products generated utilizing the synthesis method described in Claim 1 which includes the production of preparations with the general composition of pharmaceutical-pharmaceutical, molecule-molecule, and pharmaceutical-molecule. The end-product analogs are created through the generation of covalent bond structures at amines (e.g. amine-reactive N-hydroxysuccinimide ester analogs), hydroxyls (e.g. hydroxyl-reactive isocyanate analogs), phosphates (e.g. phosphate-reactive carbodiimide analogs), carboxyls (e.g. carboxyl-reactive carbodiimide analogs), carbonyls (e.g. carbonyl-reactive hydrazides), and/or sulfhydryls (e.g.

sulfhydryl-reactive maleimide analogs) within the chemical composition of the pharmaceuticals or molecular entitities applied. Reaction end-products are synthesized with a single molecule/pharmaceutical precursor (e.g. gemcitabine-gemcitabine) or two different molecular/pharmaceutical precursors (e.g. gemcitabine-decitabine) in combination with a covalent bond forming reagent (FIGS. 4, 5a, 5b & 5c & 6). Examples of relevant pharmaceuticals used in synthetic organic chemistry reactions includes but is not restricted to the anthracyclines, gemcitabine, eribulin, cytosine arabinoside (ara-C, cytarabine), 6-thioguanine, fludarabine (Fludara), 5-azacytidine, decitabine, lenalidomide, temozolomide, steroids (e.g. dexamethazone), and phosphated steroid analogs (e.g. dexamethazone phosphate).

Reaction end-products generated utilizing the synthesis method described in Claim 2 which includes analogs consisting of pharmaceuticals (or molecules that possess or will be capable of evoking a biological or chemical response of therapeutic benefit in their final form) that are covalently bound to large molecular weight platforms including but not limited to immunoglobulin (IgG), immunoglobulin fragments (e.g. Fab′), receptor ligands (e.g. EGF, VEGF, IGF), receptor ligand fragments (e.g. EGF, VEGF, IGF), natural ligands (e.g. lectins, glycoproteins, carbohydrates), and synthetic molecules (e.g. carbohydrates, aminated carbohydrates, partially oxidized carbohydrates, peptides, nucleotide sequences like siRNA). Pharmaceuticals (or other molecules capable of imparting chemical properties or biological activity in their final form) are covalently bound to large molecular weight platforms like immunoglobulin at chemical groups embedded within their structure that can include but not be limited to amines, hydroxyls, carboxyls, carbonyls, and/or sulfhydryls when applied in combination with a corresponding covalent bond-forming reagent (FIGS. 1 & 2). Covalent bond-forming reagents can possess a chemical group that includes but is not restricted to amine-reactive N-hydroxysuccinimide esters, hydroxyl-reactive isocyanates, carboxyl-reactive carbodiimides, carbonyl-reactive hydrazides, or sulfhydryl-reactive maleimides in addition to a UV-light activated diazirine (e.g. azipentanoate) chemical group (FIGS. 1 & 2). The resulting UV-photoactivated pharmaceutical intermediate is covalently bonded to large molecular weight platforms (e.g. immunoglobulin, receptor ligands) at various amino acid residues though exposure to UV-light at a specific wavelength. Examples of relevant pharmaceuticals includes but is not restricted to the anthracyclines, gemcitabine, eribulin, cytosine arabinoside (ara-C, cytarabine), 6-thioguanine, fludarabine (Fludara), 5-azacytidine, decitabine, lenalidomide, and temozolomide. Examples of relevant end-products include but is not limited to epirubicin-(C₃-amide)-[anti-HER2/neu or anti-EGFR] and gemcitabine-(C₄-amide)-[anti-HER2/neu or anti-EGFR] (FIGS. 1 & 2).

Reaction end-products generated utilizing the synthesis method described in Claim 3 which includes analogs that consist of a pharmaceutical (or molecule capable of evoking a biological or chemical response of therapeutic benefit) covalently linked to a large molecular weight platform that includes but is not limited to immunoglobulin, immunoglobulin fragments (e.g. Fab′), receptor ligands (e.g. EGF, VEGF, IGF), receptor ligand fragments (e.g. EGF, VEGF, IGF), natural ligands (e.g. lectins, carbohydrates, glycoproteins), and synthetic molecules (e.g. aminated carbohydrates, partially oxidized carbohydrates, peptides, nucleotide sequences like siRNA). Pharmaceuticals or other molecules capable of imparting chemical properties or biological activity are covalently bonded to large molecular weight platforms like immunoglobulin at a phosphate chemical group embedded within their chemical structure when utilized in combination with a covalent bond forming reagent that contain a phosphate-reactive carbodiimide group (supplemented with an imidazole analog) (FIG. 3). Pharmaceuticals (or other molecules that possess or will impart biological or chemical activity) in the form of a chemically reactive pharmaceutical intermediate is covalently bound to large molecular weight platforms (e g immunoglobulin, receptor ligands) at ε-amines of lysine residues, or hydroxyls of serine residues, or carboxyls of glutamate or aspartate residues, or sulfhydryls of cysteine residues found within the amino acid sequence of polypeptide proteins (e.g. natural or synthetic) or aminated carbohydrates/glycoproteins. Carbonyl groups of partially oxidized carbohydrates or glycoproteins are also included in this context. Examples of relevant pharmaceuticals include but is not be restricted to fludarabine (Fludara), clindamysin phosphate and phosphated steroid-core analogs (e.g. dexamethazone phosphate) in the form of fludarabine-(C₅-phosphophenooxyl)-[anti-HER2/neu or anti-EGFR], dexamethazone-(C₂₁-phosphophenooxyl)-[anti-HER2/neu or anti-EGFR], and clindamysin-(C₂-phosphophenooxyl[anti-HER2]neu or anti-EGFR) (FIG. 3).

Additive and synergistic biological, potency or efficacy is potentially achieved through a combination of [i] dual pharmaceutical mechanisms of action for two different pharmaceutical agents; [ii] dual biological effect of the pharmaceutical agents in combination with the inhibitory properties of the large molecular weight binding ligand (e.g. trophic receptor inhibition by anti-HER2/neu, anti-EGFR, anti-VEGFR immunoglobulins); [iii] selective “targeted” delivery, progressive cellular deposition, and elevated cytosol accumulation; [iii] in-vivo antibody dependent cell cytotoxicity and complement-mediated cytolysis at immunoglobulin “target” sites. Relevant examples in this regard include epirubicin-(C₃-amide)-[anti-HER2/neu or anti-EGFR], epirubicin-(C₁₃-imino)-[anti-HER2/neu or anti-EGFR], gemcitabine-(C₅-carbamate)-[anti-HER2/neu or anti-EGFR], and gemcitabine-(C₄-amide)-[anti-HER2/neu or anti-EGFR] (FIGS. 1 & 2).

The above detailed description is presented to enable any person skilled in the art to make and use the invention. Specific details have been revealed to provide a comprehensive understanding of the present invention, and are used for explanation of the information provided. These specific details, however, are not required to practice the invention, as is apparent to one skilled in the art. Descriptions of specific applications, analyses, and calculations are meant to serve only as representative examples. Various modifications to the preferred embodiments may be readily apparent to one skilled in the art, and the general principles defined herein may be applicable to other embodiments and applications while still remaining within the scope of the invention. There is no intention for the present invention to be limited to the embodiments shown and the invention is to be accorded the widest possible scope consistent with the principles and features disclosed herein.

While various embodiments of the present invention have been described above, it should be understood that they have been presented by way of example and not limitation. It will be apparent to persons skilled in the relevant art(s) that various changes in form and detail can be made therein without departing from the spirit and scope of the present invention. In fact, after reading the above description, it will be apparent to one skilled in the relevant art(s) how to implement the invention in alternative embodiments. Thus, the present invention should not be limited by any of the above-described exemplary embodiments.

The apparatus, processes, methods, and system of the present invention are often best practiced by empirically determining the appropriate values of the operating parameters, or by conducting simulations to arrive at best design for a given application. Accordingly, all suitable modifications, combinations, and equivalents should be considered as falling within the spirit and scope of the invention.

What is claimed is:

1. A method of single- or multi-phase organic chemical reaction for the synthetic production of covalent pharmaceutical-receptor ligand, pharmaceutical-immunoglobulin, pharmaceutical-immunoglobulin fragment, pharmaceutical-synthetic ligand, multi pharmaceutical-pharmaceutical complex, pharmaceutical-molecule, and molecule-molecule analogs, and fragments thereof, that may possess selective targeted delivery properties for use in fighting specific diseases including cancer/neoplastic conditions, auto-immune disorders, and organ-transplant rejection in humans and animals in need thereof, the method comprising:
forming a reaction mixture by combining at least one covalent bond-forming reagent containing at least one amine-reactive chemical group (e.g. N-hydroxysuccinimide esters) in addition to at least one ultraviolet light activated diazirine chemical group, or at least one phosphate reactive group (e.g. 1-ethyl-3-(3-dimethylaminopropyl) carbodiimide with imidazole), or at least one carboxyl reactive group (e.g. 1-ethyl-3-(3-dimethylaminopropyl) carbodiimide with N-hydroxysuccinimide), or at least one hydroxyl reactive group, or at least one sulfhydryl reactive group, or at least one carbonyl reactive group, and
optionally utilizing at least one reactive intermediate synthesized as a precursor in the reaction mixture.

2. A method of single- or multi-phase organic chemical reaction for the synthetic production of covalent pharmaceutical-receptor ligand, pharmaceutical-immunoglobulin, pharmaceutical-immunoglobulin fragment, pharmaceutical-synthetic ligand, multi pharmaceutical-pharmaceutical complex, pharmaceutical-molecule, and molecule-molecule analogs, and fragments thereof, that may possess selective targeted delivery properties for use in fighting specific diseases including cancer/neoplastic conditions, auto-immune disorders, and organ-transplant rejection in humans and animals in need thereof, the method comprising:
forming a reaction mixture by combining at least one covalent bond-forming reagent containing at least two amine-reactive chemical groups (e.g. dual N-hydroxysuccinimide esters (disuccinimidyl glutarate, disuccinimidyl suberate, disuccinimidyl tartarate); dual imidoesters (dimethyl adipimidate, dimethyl pimelimidate) and similar dual amine reactive reagents (1,5-Difluoro-2,4-dinitrobenzene)); and
optionally utilizing at least one reactive intermediate synthesized as a precursor in the reaction mixture.

3. The method of claims 1 or 2, wherein the therapeutic/diagnostic/pharmaceutical agent used in forming the reaction mixture is selected from a group including the anthracyclines, gemcitabine, gemcitabine phosphate, eribulin, cytosine arabinoside, 6-thioguanine, fludarabine, fludrabine phosphate, 5-azacytidine, decitabine, clofarabine, cladribine, lenalidomide, temozolomide, steroids, phosphated steroid analogs, non-steroidal anti-inflammatory agents, photodynamic agents, or a combination thereof.

4. The method of claims 1 or 2, wherein the therapeutic/diagnostic/pharmaceutical agent contains at least one amine, hydroxyl, carboxyl, carbonyl, phosphate, or sulfhydryl chemical group.

5. The method of claim 1, wherein the therapeutic/diagnostic/pharmaceutical agent is covalent bound to a second molecule that contains at least one amine, hydroxyl, carboxyl, carbonyl, phosphate, or sulfhydryl chemical group.

6. The method of claim 1, wherein the covalent bond-forming reagent contains at least one amine-reactive chemical group compound in addition to at least one ultraviolet light reactive chemical group compound, or at least one phosphate reactive chemical group, or at least on carboxyl reactive chemical group, or at least one carbonyl reactive chemical group, or at least one sulfhydryl reactive chemical group, or at least one hydroxyl reactive chemical group.

7. The method of claim 2, wherein the covalent bond-forming reagent contains at least two of the same chemically-reactive group compounds selected from the group consisting of amine-reactive, hydroxyl-reactive, phosphate-reactive, carboxyl-reactive, carbonyl-reactive, or sulfhydryl-reactive.

8. The method of claim 1, wherein the covalent bond-forming reagent contains at least two different chemically-reactive group compounds selected from the group consisting of amine-reactive, hydroxyl-reactive, phosphate-reactive, carboxyl-reactive, carbonyl-reactive, or sulfhydryl-reactive.

9. The method of claim 1, wherein the covalent bond-forming reagent initially reacts with a phosphate group compound, or a carboxyl group compound, or a combination thereof, and subsequently reacts with at least one amine group compound.

10. The method of claim 5, wherein the analog consists of a therapeutic/diagnostic pharmaceutical covalently linked to a large molecular weight platform selected from the group consisting of immunoglobulin, immunoglobulin fragments, receptor ligands, receptor ligand fragments, natural ligands, synthetic ligands, lectins, and other natural or synthetic molecules.

11. The method of claims 1 or 2, further comprising applying at least one aqueous solvent, at least one organic solvent, or a combination thereof, to the reaction mixture.

12. The method of claim 6, wherein the covalent bond-forming reagent is reacted with a corresponding amine, hydroxyl, carboxyl, carbonyl, phosphate, or sulfhydryl group of a therapeutic/diagnostic pharmaceutical for creating a covalent bond structure and the other second chemically active group (ultraviolet-activated, amine reactive, carboxyl reactive, phosphate reactive, sulfhydryl reactive, carbonyl reactive, hydroxyl reactive) is covalently bound to a large molecular weight platform following activation.

13. The method of claim 12, wherein the large molecular weight platform is selected from the group consisting of immunoglobulin, immunoglobulin fragments, receptor ligands, receptor ligand fractions, natural ligands, synthetic ligands, lectins, and other natural molecules, or synthetic ligand analogs.

14. A method of single-phase organic chemical synthesis for the synthetic production of covalent pharmaceutical-receptor ligand, pharmaceutical-immunoglobulin, pharmaceutical-synthetic ligand, pharmaceutical-pharmaceutical, pharmaceutical-molecular, and molecular-molecular analogs, and fragments thereof, that possess selective delivery properties for use in fighting specific diseases including cancer/neoplastic disease, autoimmune disorders, severe inflammatory reactions, and organ transplant rejection in humans and animals in need thereof, the method comprising:
forming a reaction mixture by combining at least one amine, hydroxyl, carboxyl, carbonyl, phosphate, or sulfhydryl chemical group compound with at least one covalent bond-forming reagent containing at least two chemically reactive groups that form covalent bonds at specific chemical groups; and
optionally utilizing at least one reactive intermediate synthesized as a precursor in the reaction mixture.

15. The method of claim 14, wherein the therapeutic/diagnostic pharmaceutical agent used in forming the reaction mixture is selected from the group consisting of anthracyclines, gemcitabine, gemcitabien phosphate, eribulin, cytosine arabinoside, 6-thioguanine, fludarabine, fludarabine phosphate, clofarabien, cladribine, 5-azacytidine, decitabine, lenalidomide, temozolomide, steroids, phosphated steroid analogs, non-steroidal anti-inflammatory agents, photodynamic agents, or a combination thereof.

16. The method of claim 14, wherein the two chemically reactive group compounds (covalent bond forming agents) comprise amine-reactive ester groups.

17. The method of claim 14, wherein the covalent bond-forming reagent contains at least two sulfhydryl reacting groups.

18. The method of claim 14, further comprising applying at least one aqueous solvent, at least one organic solvent, or a combination thereof, to the reaction mixture.

19. The method of claim 14, wherein the covalent bond-forming reagent is reacted with a corresponding amine, hydroxyl, carboxyl, carbonyl, phosphate, or sulfhydryl group of a therapeutic/diagnostic pharmaceutical for creating a covalent bond structure and the ultraviolet-activated pharmaceutical is covalently bound to a large molecular weight platform by exposure to ultraviolet light.

20. The method of claim 19, wherein the large molecular weight platform is selected from the group consisting of immunoglobulin, immunoglobulin fragments, receptor ligands, receptor ligand fractions, natural ligands, synthetic molecules, or synthetic ligand analogs.

21. A method of single- or multi-phase organic chemical synthesis for the synthetic production of covalent pharmaceutical-receptor ligand, pharmaceutical-immunoglobulin, pharmaceutical-synthetic ligand, pharmaceutical-pharmaceutical, pharmaceutical-molecular, and molecular-molecular analogs, and fragments thereof, that possess selective delivery properties for use in fighting specific diseases including cancer/neoplasia, severe inflammatory reactions, auto-immune disorders, or organ-transplant rejection in humans and animals in need thereof, the method comprising:
forming a reaction mixture by combining at least one amine, hydroxyl, carboxyl, carbonyl, phosphate, or sulfhydryl chemical group compound with at least one covalent bond-forming reagent containing at least two different chemically reactive group compounds that form covalent bonds with two different chemical groups; and
optionally utilizing at least one reactive intermediate synthesized as a precursor in the reaction mixture.

22. The method of claim 21, wherein the covalent bond-forming reagent contains at least one amine-reactive analog, hydroxyl-reactive analog, phosphate-reactive analog, carboxyl-reactive analog, carbonyl-reactive analog, or sulfhydryl-reactive analog, or a combination thereof.
